(19) United States
(12) Patent Application Publication
(10) Pub. No.: US 2021/0267858 A1
(43) Pub. Date: Sep. 2, 2021

(54) WATER-FREE TOPICALLY APPLICABLE PREPARATION

(71) Applicant: BEIERSDORF AG, Hamburg (DE)

(72) Inventors: Manuela KOEHLER, Hamburg (DE); Sabilla ZHONG, Hamburg (DE); Christina JAPP, Hamburg (DE); Doreen GUTZKE, Bispingen (DE); Dominik GOEDDERTZ, Hamburg (DE); Svea WISCHHOEFER, Hamburg (DE)

(21) Appl. No.: 16/973,036

(22) PCT Filed: May 6, 2019
(86) PCT No.: PCT/EP2019/061574

§ 371 Date: Dec. 8, 2020

(30) Foreign Application Priority Data
Jun. 19, 2018 (DE) 10 2018 209 853.0

(51) Int. Cl. A61K 8/37; A61K 8/92; A61K 8/73; A61K 8/36; A61K 8/891; A61K 8/06; A61Q 19/00

(52) U.S. Cl. CPC A61K 8/37; A61K 8/922; A61K 8/731; A61K 8/732; A61K 8/361; A61K 2800/594; A61K 8/064; A61Q 19/00; A61K 2800/31; A61K 2800/546; A61K 2800/10; A61K 8/891

(57) ABSTRACT

A water-free preparation comprising one or more lipids, at least one w/o emulsifier and at least 0.05% by weight of one or more superabsorbents, allows the simultaneous drying and the skin care after washing, bathing or showering in one step.

The invention is a topically applicable preparation which can advantageously be applied and distributed on moist or wet skin. The wetness/moisture is absorbed and is transformed directly into skin care. Advantageously, the skin does not then have to be dried off.

The water-free preparation comprises one or more lipids, at least one W/O emulsifier and at least 0.05% by weight of one or more superabsorbents.

In daily skin care and routine activities, human skin is regularly wet or damp. Be it after washing, showering, bathing, swimming or through sweat, frequent drying of the skin is a result.

On the one hand, the consumer wants to avoid or reduce the constant drying, since this also results in the skin drying out, and on the other hand, the consumer also wants to care for the skin.

In a common routine, after skin cleansing, the skin is dried and then a skin care product is applied. If the skin is wet or damp with sweat, the sweat is often wiped off before a skin care product, for example a sunscreen product, is then applied.

Avoiding these circumstances or reducing the time required for this purpose would be desirable.

It is known that the majority of skin care products contain water. This makes the storage and transportation of skin care products difficult.

There are therefore so-called instant cosmetics, water-free cosmetics which can be used without water or which are only mixed with water prior to use.

In the meantime, due to the regulation on air travel that does not allow liquids over 100 ml to be carried in hand luggage, there are also many cosmetic products that make do without water.

Anhydrous cosmetics also have the advantage that the products require fewer or no preservatives. Cosmetics without water also have an advantage when it comes to packaging, because they usually require less material.

It is therefore desirable to provide a product which demonstrates the advantages presented and avoids the disadvantages of the prior art.

EP 2023888 A2 discloses cosmetic water-containing preparations comprising superabsorbents based on starch.

FR 2999919 describes 0/W and W/O emulsions with superabsorbents and siloxane elastomers.

US 2013338052 describes water-containing formulae with superabsorbent particles.

So-called in-shower products, which can be applied to wet skin, are also in the prior art. EP 2819639 A2 describes such products.

After applying in-shower products to wet skin, the skin is rinsed off and one still has to dry oneself off.

A property of cosmetic products that is essential for the consumer, but difficult to measure quantitatively, is their texture and sensory properties. The term “texture” is understood to mean those properties of a cosmetic which are due to the microstructure of the preparation, which can be felt, inter alia, by tactile senses and touch, and possibly may be expressed in mechanical or rheological flow properties. The texture can be tested in particular by means of sensors. The texture of cosmetic products, which can optionally be influenced with the aid of additives, is of almost the same importance for the consumer as their objectively identifiable effects.

The term “sensory” is used to describe the scientific discipline that deals with the evaluation of cosmetic preparations on the basis of sensory impressions. The sensory evaluation of a cosmetic is based on visual, olfactory and haptic impressions.
visual impressions: all features that can be perceived by eye (color, shape, structure).
olfactory impressions: all odor impressions perceptible when air is drawn in through the nose, which can often be differentiated into initial odor (top note), main odor (middle note, body) and post odor (finish). The volatile substances only released during application also contribute to the olfactory impression.
haptic impressions: all sensations of the sense of touch that primarily concern the structure and consistency of the product.

The sensory analysis makes use of the possibility of integrally recording the overall sensory impression of a product. Disadvantages of sensory analysis are the subjectivity of the impression, the fact that the test subjects can be easily influenced and the resulting high variability of the results. These weaknesses are countered today by the use of groups of trained test subjects, mutual shielding of the testers and statistical evaluation of the usually abundant analysis data.

It is therefore an object of the present invention to provide preparations which, in addition to the criteria customary for cosmetics such as compatibility, storage stability and the like, also offer cosmetic, in particular sensory, benefits hitherto unknown to the consumer.

Surprisingly, the problems and objects presented are achieved by the preparations according to the invention and uses thereof.

The invention comprises in its first embodiment a water-free topically applicable preparation comprising
a) one or more lipids,
b) at least one W/O emulsifier and
c) at least 0.05% by weight of one or more superabsorbents, based on the total mass of the preparation.

Preparations that can be applied topically are, in particular, cosmetic and dermatological preparations, such as gels, ointments, creams, lotions, but also preparations in the form of aerosols or pump sprays, and impregnation media for cloth, pad or plaster application. The preparation can be liquid, semi-solid or solid.

As a preferred embodiment, the water-free preparation only comprises one or more lipids, at least one W/O emulsifier and at least 0.05% by weight of one or more superabsorbents.

In the simplest embodiment, the preparation comprises only one lipid, one W/O emulsifier and one superabsorbent polymer.

Preferred, advantageous and more pleasant to the senses is an embodiment in which the preparation comprises a combination of at least two oils, two W/O emulsifiers and one superabsorbent.

If the preparation according to the invention is applied to a moist surface, it is advantageous and extremely astonishing that the water is absorbed in a few seconds and thus “drying” takes place in a very short time.

With the preparation according to the invention, a novel technology for appropriate skin care has been invented, which enables both convenience and benefit for the consumer in terms of daily body care.

The preparation according to the invention enables simultaneous drying and skin care in one step after washing, bathing or showering.

The preparation according to the invention can be applied to still wet or damp skin and spread, and then the skin is in the same state as if you had dried yourself off, for example with a towel, and only then applied a skin care product.

When the preparation according to the invention is used, the water, wetness, moisture on the skin after washing, showering, bathing or sweating is immediately converted into skin and body care.

The advantages of the new technology are many and varied, and some examples are given below:
the use of a towel after washing, bathing or showering is superfluous
the frequency of washing towels is reduced
time saving; while drying, cream is applied to the skin at the same time; the water and moisture on the skin is used
reduction in product size and volume; there is no water normally present in cosmetics. A type of concentrate can be used and transported. Smaller containers are required and thus less waste is produced. Less fuel is used per unit of consumption during transport.
individualization of bathing/showering or skin care. Men or women can control themselves what kind of product is applied to the skin by leaving more or less water on the skin. There are no disadvantages even if the product according to the invention is used on dry skin. It then feels richer because it is not diluted with water.
high skin moisturizing performance
the preparation according to the invention opens up multiple benefits in terms of galenics since, due to the absence of water, for example, water-sensitive substances can be easily incorporated and in different amounts
preservatives can be omitted
irritated skin no longer has to be rubbed uncomfortably with a towel and is not still further irritated
even UV protection can be applied to wet skin in this way
even UV protection can be applied to wet skin in this way, even if what is wanted is to shower and dry off once again, so-called in-shower application
in this way even waterproof UV protection can be offered, even without film formers
on the beach, it is not necessary to dry yourself off with a sandy towel before applying the product (UV protection or after sun)

Compared to the preparations of the prior art which comprise superabsorbents, such as FR 2999919 for example, the preparations according to the invention are advantageously not emulsions and, in addition, the preparations according to the invention are free of water.

The inventive constituents of the topically applicable preparation and also optionally possible constituents are preferably selected from the groups detailed below.

### a) Lipids

In the context of the present disclosure, the term “lipids” is used as a generic term for fats, oils, waxes and the like, as is familiar to the person skilled in the art. The terms “oil phase” and “lipid phase” are also used synonymously.

The preparation according to the invention comprises one or more lipids.

Preference is given to lipids having a low viscosity.

Preference is given to lipids having a high spreading value.

Preference is given to lipids having a low viscosity and a high spreading value.

The viscosity (25° C., dynamic) of the lipids is therefore preferably below 250 mPas and the spreading value is advantageously above 400.

Rotational rheometers, inter alia, are available for measuring the viscosity of cosmetic preparations.

The viscosity is measured with a rotational rheometer by rotating a measurement body in the substance to be measured. The viscosity of the substance is proportional to the force with which it counteracts the rotation of the measurement body. For instance, the viscosity can be determined using a Viscotester VT-02 from Haake at 25° C. with the aid of rotating body 1 or 2 and reading the scale 1 or 2.

The spreading value is measured, for example, as follows:

The lipids are measured at room temperature. A measurement takes 10 minutes per filter paper. Round Whatman 589/2 110 mm ashless/white ribbon filter paper can be used as filter paper. 25 μl of the lipid to be measured are applied, more precisely, pipetted onto the center of the filter paper from a height of ca. 30 mm. Care must be taken to ensure that the lipid is not applied in a scattered manner during application. The stopwatch is started on application.

After a spreading time of 10 minutes, the resulting spot is outlined. This measurement is carried out on five filter papers for one lipid. The diameter in mm of the outlined spot is determined at three different points. Using 15 values (5 filter papers each with 3 diameters), the mean value is determined. This mean value is then used to calculate the area of the spread lipid (calculation of the area of a circle):

A=π*r² or A=π*(d/2)²

The spreading value is specified in the unit mm²/(10 min) for 25 μL

It is therefore advantageous to choose lipids from the following table:

| | Viscosity | Spreading value |
|---|---|---|
| INCI | [mPas] | [mm²/10 min] |
| Dibutyl Adipate | 5 | 935 |
| Isopropyl Myristate | 4.6 | 707 |
| Isodecyl Neopentanoate | 3.8 | 962 |
| Octyldodecanol | 47.7 | 440 |
| Caprylic/capric triglycerides | 20 | 570 |
| Propylene Glycol Dicaprylate/Dicaprate | 12 | 868 |
| Dicaprylyl ether | 3.3 | 1020 |
| Paraffinum Liquidum | 27.4 | 550 |
| Isopropyl palmitate | 6.2 | 625 |
| C13-16 Isoparaffin | 2.6 | 1018 |
| Isohexadecane | 3.7 | 990 |
| Cyclomethicone | 5 | 804 |
| Hydrogenated Polydecene | 45.6 | 510 |
| Octyldodecyl myristate | 24.2 | 755 |
| Decyl oleate | 14.1 | 668 |
| Butylene Glycol Dicaprylate/Dicaprate | 9.6 | 813 |
| Dicaprylyl Carbonate | 6.3 | 875 |
| Hydrogenated Polyisobutene | 34.1 | 616 |
| Isopropyl Isostearate | 9.5 | 790 |
| Cetearyl Isononanoate | 14.9 | 780 |
| Isopropyl Stearate | 6.8 | 910 |
| Phenyl Trimethicone | 17.3 | 631 |
| C12-15 Alkyl Benzoate | 12.2 | 730 |
| Ethylhexyl Cocoate | 7.9 | 930 |
| Squalane | 30 | 594 |
| Ethylhexyl Stearate | 29 | 697 |
| Cetearyl Ethylhexanoate + Isopropyl | 24 | 683 |
| Myristate | | |
| C12-13 Alkyl Lactate | 17 | 800 |
| Coco-Caprylate/Caprate | 5 | 755 |
| Persea Gratissima Oil | 180 | 416 |
| Simmondsia Chinensis Seed Oil | 35.8 | 449 |

The proportion of one or more lipids is advantageously selected in the range from 25% by weight to 65% by weight, preferably in the range from 35% by weight to 55% by weight, particularly preferably in the range from 30% by weight to 50% by weight, based on the total mass of the preparation.

If it is a photoprotective preparation comprising appropriate UV filters, which in turn can also be understood as lipids, the proportion of lipids including any UV filters is in the range from 20% by weight to 75% by weight, preferably in the range 35% by weight to 65% by weight, particularly preferably in the range from 40% by weight to 60% by weight, based on the total mass of the preparation.

In a further embodiment, waxes are advantageously chosen as lipids. More advantageously, in addition to waxes, further lipids are also present.

In accordance with the invention, fats and fat-like substances having a waxy consistency may also be used as waxes. These include, inter alia, fats (triglycerides), mono and diglycerides, natural and synthetic waxes, silicone-based waxes, hydrocarbon waxes, fatty and wax alcohols, esters of fatty alcohols and fatty acids, and fatty acid amides or any mixtures of these substances.

The waxes are particularly preferably selected from the group of fats, in particular from the group of natural waxes, such as in particular Shorea stenoptera seed butter, hydrogenated vegetable oil, hydrogenated coco-glycerides, Butyrospermum parkii butter, Theobroma cacao (cocoa) seed butter, mango butter, hydrogenated palm kernel glycerides, hydrogenated palm glycerides, sunflower seed wax, soybean glycerides, Butyrospermum parkii unsaponifiables, hydrogenated castor oil, wool wax, Cera alba, beeswax, sugar cane wax, Cera carnauba, candelilla wax, japan wax, hydrogenated rapeseed oil, shellac wax, hydrogenated soybean oil.

Selected in particular from the group of synthetic waxes are Cera microcristallina, synthetic beeswax, synthetic wax, polyethylene, paraffin wax, ceresin, ozokerite.

Selected in particular from the group of fatty acids are palmitic acid and stearic acid.

Selected in particular from the group of esters of fatty acids are cetearyl nonanoate, methyl palmitate, glyceryl tribehenate, glyceryl laurate, glyceryl stearate, cetyl palmitate; shea butter oleyl esters, PEG-8 beeswax.

The fatty alcohols used are preferably C14 to C22 fatty alcohols. The fatty alcohols are preferably selected from the group of linear fatty alcohols, in particular myristyl alcohol (C₁₄H₃₀O), cetyl alcohol (or palmityl alcohol) (C₁₆H₃₄O), stearyl alcohol (or octadecyl alcohol) (C₁₈H₃₈O) and cetyl stearyl alcohol (cetearyl alcohol), behenyl alcohol, lanolin alcohol, a mixture of the alcohols cetyl alcohol (hexadecanol) and stearyl alcohol (octadecanol).

In accordance with the invention, waxes are preferably selected having a melting point in the range between 30°-120°. Particularly preferred are waxes having a melting point in the range of 40° C. to 100° C., particularly preferably in the range of 40° C. to 90° C.

The preferred melting point arises on the one hand from the body temperature, since the lipids should not melt thereat (lower limit), and at the same time still be easy to process (upper limit).

The preferred waxes mentioned have a positive effect on the sensory properties of the preparation according to the invention.

In contrast to “oily”, “waxy” feels more dry and rough and therefore results in a more attractive product.

The proportion of one or more waxes is advantageously selected in the range from 1% by weight to 20% by weight, preferably 2%-12% by weight, particularly preferably 4% by weight to 10% by weight, based on the total mass of the preparation.

b) W/O Emulsifiers The preparation according to the invention comprises one or more water-in-oil emulsifiers.

W/O emulsifiers according to the invention advantageously bind the water in the oil phase and thus support the drying properties.

In comparison, O/W emulsifiers show that the water accumulates in the outer phase with the result that the skin feel retains a wet, moist feeling, so that the purpose according to the invention is not met.

The drying effect according to the invention was likewise not achieved in a W/O/W emulsion.

It is preferable to select two W/O emulsifiers, since even better sensory properties and, above all, faster drying kinetics are thus achieved. Furthermore, the appearance can be controlled during distribution.

In accordance with the invention, the W/O emulsifiers also include the group of W/Si emulsifiers.

In general, emulsifiers having an HLB value of up to ca. 8 are considered to be W/O emulsifiers. In contrast, O/W emulsifiers have HLB values of greater than 8 to 15. Substances having HLB values greater than 15 are often referred to as solubilizers. In the prior art, W/O emulsions are sometimes stabilized by adding O/W emulsifiers (HLB>8) which function as a co-emulsifier. Dispensing with such substances reduces the complexity of the raw materials and prevents the formulation from flipping into the undesired 0/W range. In accordance with the invention, it is advantageous to dispense with adding emulsifiers having an HLB>8.

In the preparation according to the invention, a W/O emulsifier, which can be selected from the list of known W/O emulsifiers, serves as the main emulsifier. The W/O emulsifier or emulsifiers preferably have an HLB value of less than or equal to 8.

One or more W/O emulsifiers that may be used advantageously can be selected from the group comprising polyglyceryl-n diisostearates, polyglyceryl-n dipolyhydroxystearates, particularly polyglyceryl-n dipolyhydroxystearates, diisostearoyl polyglyceryl-n dimer dilinoleates and/or polyglyceryl-n diisostearates/polyhydroxystearates/sebacates.

One or more advantageous W/O emulsifiers can be selected from the group polyglyceryl-3 diisostearate, polyglyceryl-2 dipolyhydroxystearate, diisostearoyl polyglyceryl-3 dimer dilinoleate and/or polyglyceryl-4 diisostearate/polyhydroxystearate/sebacate.

The proportion of the total amount of W/O emulsifiers is preferably 0.25 to 14% by weight, in particular up to 10% by weight, particularly 0.75 to 7% by weight, based on the total mass of the preparation.

Preference is given to the combination of 5% by weight polygylceryl-3 diisostearate and 2% by weight polyglyceryl-2 dipolyhydroxystearate.

In accordance with the invention, particular preference is given to the combination of diisostearoyl polyglyceryl-3 dimer dilinoleate and polyglyceryl-4 diisostearate/polyhydroxystearate/sebacate in the ratio from 2:1 to 1:2, particularly preference being given to the ratio 1:1.

In accordance with the invention, particular preference is given to the combination of 0.25-1.5% by weight diisostearoyl polyglyceryl-3 dimer dilinoleate and 0.25-1.5% by weight polyglyceryl-4 diisostearate/polyhydroxystearate/sebacate.

The preferred emulsifiers mentioned achieve good results with regard to the feeling of drying, sensory properties (lightness) and price.

By way of preference, the proportion of polyglyceryl-3 diisostearate is selected in the range from 2 to 7% by weight, in particular 5% by weight, the proportion of polyglyceryl-2 dipolyhydroxystearate is selected in the range from 1 to 3% by weight, in particular 2% by weight, the proportion of diisostearoyl polyglyceryl-3 dimer dilinoleate is selected in the range from 0.5 to 2% by weight, in particular 1% by weight, and the proportion of polyglyceryl-4 diisostearate/polyhydroxystearate/sebacate is selected in the range 0.5 to 2.5% by weight, in particular 1% by weight, based in each case on the total mass of the preparation.

Particular preference can be given to selecting the system of 1% by weight diisostearoyl polyglyceryl-3 dimer dilinoleate and 1% by weight polyglyceryl-4 diisostearate/polyhydroxystearate/sebacate. In the case of a UV filter-containing formula, particular preference can be given to selecting the system of 0.5% by weight diisostearoyl polyglyceryl-3 dimer dilinoleate and 0.5% by weight polyglyceryl-4 diisostearate/polyhydroxystearate/sebacate.

The proportions by weight refer in each case to the total mass of the preparation.

### c) Superabsorbents

The preparation according to the invention comprises at least 0.05% by weight of one or more superabsorbents, based on the total mass of the preparation.

For superabsorbents, terms such as “highly swellable polymer”, “hydrogel”, “hydrogel-forming polymer”, “water-absorbent polymer”, “absorbent gel-forming material”, “swellable resin”, “water-absorbent resin”, “gelling agent” or similar terms are commonly used. These are primarily crosslinked hydrophilic polymers, in particular polymers composed of (co)polymerized hydrophilic monomers, graft (co)polymers of one or more hydrophilic monomers on a suitable graft base, crosslinked natural carbohydrates, for example cellulose ethers or starch ethers, crosslinked carboxymethyl cellulose and carboxymethyl starches (sodium starch glycolates or sodium cellulose glycolates), partially crosslinked polyalkylene oxide or natural products that swell in aqueous liquids, such as guar derivatives, with water-absorbing polymers based on partially neutralized acrylic acid being the most common. Superabsorbents are materials that can absorb many times their mass of liquids. If the surface of the superabsorbent is also cross-linked, a certain retention can even be obtained, that is to say that it does not release absorbed fluids again, even under mechanical stress. Such liquids to be absorbed can be water or aqueous solutions and organic liquids and mixtures thereof. In the case of aqueous solutions, the superabsorbents consist in particular of crosslinked macromolecules having ionic and/or polar groups. Since the beginning of the seventies, high-performance and versatile superabsorbents have been developed based on neutralized, cross-linked polyacrylic acid (PANV).

In accordance with the invention, superabsorbents (SA) are preferably understood to mean polymers with the following chemical composition:

fully synthetic:
cross-linked polyacrylates and derivatives thereof (for example methacrylates)
polyvinyl acetate-acrylic acid copolymers
polyvinyl alcohol copolymers
polyethylene oxide
isobutylene-maleic acid copolymers
hydrolyzed polyacrylonitrile
   semi-synthetic:
starch-polyacrylate graft copolymers
hydrolyzed starch-polyacrylonitrile graft copolymers
carboxymethyl cellulose
carboxymethyl starches (also known as starch glycolates)
   natural:
alginates

By far the most important field of application of superabsorbents is the absorption of body fluids. Superabsorbents are used, for example, in diapers for small children, incontinence products for adults, feminine hygiene products or in wound care. Other areas of application of superabsorbents are in agricultural horticulture, as water storage for protection against fire, for the absorption of liquids in food packaging or, more generally, for the absorption of moisture.

The use of superabsorbents is rarely observed in cosmetics. Products are only known in which a “sorbet-like” surface can be produced by using polyacrylate superabsorbents.

Polar polymers, such as polyacrylic acid for example, already bind large amounts of the likewise polar water (1000 times the mass) in its interior and swell in this process.

Due to the sodium ions within the polymer structure, the molecule also develops a strong salt character. Further absorption of fluid occurs due to osmotic pressure.

This osmotic pressure ultimately determines the absorption capacity of the superabsorbent.

This results in the following relationships: The more sodium ions are incorporated into the polymer, the greater the amount of liquid that can be absorbed. The higher the concentration of salts in the liquid, the lower the absorption will be. This explains that pure water is absorbed at an order of magnitude of several hundred times the weight of the superabsorbent, whereas salt solutions, such as urine etc., significantly limit the capacity of the superabsorbent. That is why only about 100 fold is absorbed from a physiological saline solution.

The principle described works not only with sodium but also with other cations.

The operating principle also explains why non-polar liquids such as oils cannot be absorbed.

From the group of fully synthetic superabsorbents, the following can preferably be selected in accordance with the invention:
crosslinked polyacrylates, especially sodium polyacrylate crosspolymer-2 (Aquakeep 10SH-NFC)
crosslinked polyacrylamides
crosslinked polymethacrylates
polyvinyl ethers
polyvinylpyridines
polyvinyl morpholinones,
N,N-dimethylaminoethyl or N,N-diethylaminopropyl acrylates and methacrylates
poloxamers
polyvinyl alcohols
polyvinyl alcohol-polyethylene glycol graft copolymers
maleic anhydride/isobutene copolymers
polyvinyl polypyrrolidones
poly(vinyl glyoxylic acid)
acryloacrylamide/sodium acrylate copolymers
starch/acrylamide/sodium acrylate copolymers

From the group of superabsorbents based on polysaccharides, the following are preferred in accordance with the invention:
sodium polyacrylate starch
alginates
alginate esters
hydroxyethyl cellulose
hydroxypropyl cellulose
hydroxypropyl methylcellulose
carboxyalkyl cellulose
sodium carboxymethyl cellulose
croscarmellose sodium
sodium starch glycolate (Glycolys, Explotab, Vivastar, Primojel)
glucomannans
galactomannans (e.g. from Konjac)
xyloglucan
(modified) cellulose fibers
cotton fibers
(modified) starches
xanthan gum
cellulose gum
carrageenan gum
guar gum
arabic
tragacanth
pustulan
callose
laminarin
guluronic acid
pullula lichenin
amylose
amylopectin
dextran
pectin
inulin
chitin
hyaluronic acid

From the group of superabsorbents based on amino acids, the following are preferably to be selected in accordance with the invention:
polyaspartic acid
polyglutamic acid
poly(ε-L-lysine)
gelatin
   and also
phytocolloids

The use of superabsorbents made from renewable raw materials is advantageous since they appear less harmful to health and more environmentally friendly and also more skin-friendly.

(Poly)saccharides such as cellulose, starch, chitin and pectins are suitable as a basis for the production of superabsorbents from renewable raw materials.

Superabsorbents which are constructed based on natural carbohydrates are preferred, very particularly preferably glycolates, also known as carboxymethyl-derivatized polymers, preferably biopolymers.

The superabsorbents based on carbohydrates are to be preferred because, surprisingly, they do not show frequent disadvantages such as the formation of clumps or grains on the skin.

Preference is given in particular to carboxymethyl cellulose and carboxymethyl starches (sodium starch glycolates or sodium cellulose glycolates).

The crosslinked carboxymethyl starch preferred according to the invention ideally has a preferred particle size distribution in which at least 95% of the particles are smaller than 130 μm, preferably smaller than 120 μm. The mean particle size is between 30-70 μm.

Superabsorbents preferred according to the invention are crosslinked carboxymethyl starches (sodium starch glycolates) characterized in that they can absorb many times their weight of water, for example Explotab and Vivastarch (e.g. Explotab 3500, Explotab CLV, etc.). The water absorption capacity is accordingly a criterion for superabsorbents to be selected with preference.

Crosslinking is a process in which polymers are linked, interlinked, attached to one other and thus form a three-dimensional network. The process can take place directly during the synthesis of the polymer or subsequently.

The crosslinking can have an influence on the properties of the crosslinked polymers, such as, for example, the water absorption capacity and also the water retention.

The fact that crosslinking occurred during or after the synthesis of the polymer can also affect the properties of the final SA.

In accordance with the invention, the preparation comprises one or more water-absorbing substances which can each absorb a multiple of their weight of water.

In accordance with the invention, superabsorbents can preferably absorb up to 1000 times their weight, preferably up to 200 times their weight, of liquid. Particular preference is given to superabsorbents having a liquid absorption capacity, in particular water absorption capacity, in the range of 50 times, in particular 20 times their weight, and especially more than 5 times their weight.

In accordance with the invention, superabsorbents are therefore substances which can absorb more than up to 5 times their weight and more of liquid. In accordance with the invention, preference is given to using superabsorbents having a liquid absorption capacity of 50 up to 200 times their weight.

Preference is given to using a superabsorbent. However, depending on the desired properties, such as the sensory properties, it may be necessary to combine two or more superabsorbents.

The proportion of one or more superabsorbents according to the invention is at least 0.05% by weight, based on the total mass of the preparation. The proportion of one or more superabsorbents is preferably in the range from 0.05 to 20% by weight, preferably 0.1 to 10% by weight, based on the total mass of the preparation.

In accordance with the invention, sensory feel additives, such as aluminum starch octenyl succinates in particular, which are not superabsorbents, are preferably excluded.

Astonishingly, the superabsorbents, and here especially the preferred absorbents, do not have any negative influence on moisturizing of the skin.

As expected, it would have been feasible to use superabsorbents to remove moisture from the skin. Surprisingly, however, this was not apparent.

Skin moisture measurements were carried out according to the principle of measuring the capacitance of the skin as a dielectric medium.

For this purpose, a corneometer CM 825 from Courage 4+Khazaka (Cologne, Germany) is used. The parameters are the permittivity [a. U.], which are generated from the median of repeated measurements (10). An increase in permittivity accordingly indicates improved skin hydration.

Two preparations (652, 654) were tested, preparation 654 is non-inventive and does not comprise superabsorbent.

| A_652 | B_654 | INCI/% by weight |
|---|---|---|
| 7.21 | 7.21 | Isoamyl Laurate |
| 5 | 0 | Sodium starch glycolate |
| 3 | 3 | Hydrogenated Castor Oil |
| 22.25 | 22.25 | Isopropyl palmitate |
| 2.37 | 2.37 | Octyldodecanol |
| 2.37 | 2.37 | C12-15 Alkyl Benzoate |
| 1 | 1 | Diisostearoyl Polyglyceryl-3 Dimer Dilinoleate |
| 1 | 1 | Polyglyceryl-4 |
| | | Diisostearate/Polyhydroxystearate/Sebacate |
| 5 | 5 | Calcium Starch Octenylsuccinate + |
| | | Aqua + Calcium Chloride |
| 10 | 10 | Dimethicone + Dimethicone Crosspolymer |
| 0.8 | 0.8 | Perfume |
| 6 | 6 | Glycerol + Aqua |
| 4 | 4 | Magnesium Stearate |
| 30 | 35 | Alcohol Denat. + Aqua |

There were no significant differences in the skin moisture measurement described above between the two formulae described in the table (A_652 and B_654). It can thus be shown that the presence of a superabsorbent has no negative influence on moisturizing of the skin. Furthermore, there were no incompatibilities or reactions. No discomfort was observed.

### d.) Water-Free

“Free from” means that the proportion of the substances to be avoided is less than 1% by weight. This ensures that small amounts of these substances, which are introduced by chance or are additives related to the raw materials, are also included since they cannot exert any influence in these small amounts, in particular no influence on the properties according to the invention.

The preparation according to the invention is water-free. This means that the preparation is still considered to be free from water even if it contains small amounts of water that have been introduced or water that has no effect on the properties of the preparation.

Due to constituents of the preparation, such as skin moisturizing agents or ethanol, the water content in the preparation can be up to 1% by weight, in particular up to 0.6% by weight, or preferably less than 0.3% by weight, in particular 0% by weight, based on the total mass of the preparation, and is then still considered water-free according to the invention.

### e) UV Filters

One problem with the necessary sun protection of the skin is that the sunscreen should be spread over the entire skin. Multiple dressing and undressing is actually necessary for this purpose. The preparation according to the invention offers an ingenious simplification here. After showering or bathing, the preparation according to the invention, which comprises one or more UV filters, is applied to the bare moist or wet skin. It is not necessary to dry yourself off and the preparation can be applied directly. Then you are dry and at the same time provided with sun protection. This use is also possible on the beach with sea water.

Further advantages are:

The sun protection (SPF) when the product is applied to wet skin is comparable to the sun protection when applied to dry skin (ISO 24444: 2010 (E)).

It is particularly surprising in this case that the preparation according to the invention can be applied comfortably and without problems and that an SPF of at least 30 is achieved.

Another surprise is that the preparation according to the invention is waterproof (in accordance with Colipa 2005 water resistance), even though no film formers are present in the preparation.

Even if one feels the need to rinse off shortly after application of the preparation, a significant SPF of at least 25 still remains, according to a test design based on ISO 24444: 2010 (E). In this case, the skin is watered for 1 minute. Then, 2.0 (+/−0.05 mg)/cm2 of the preparation are applied within 30 seconds. After a waiting time of 1.5 minutes, shower off for 30 seconds and pat dry. The SPF was then measured.

In the context of the present invention, it is therefore advantageous if the preparation comprises one or more UV filters.

One or more UV filters are advantageously selected from the group of the compounds 2-phenylbenzimidazole-5-sulfonic acid and/or salts thereof; phenylene-1,4-bis(2-benzimidazyl)-3,3′-5,5′-tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 3-(4-methylbenzylidene)camphor; 3-benzylidenecamphor; ethylhexyl salicylate; terephthalidenedicamphorsulfonic acid; 2-ethylhexyl 2-cyano-3,3-diphenylacrylate; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnamate; isoamyl 4-methoxycinnamate; 2-hydroxy-4-methoxybenzophenone, 2-hydroxy-4-methoxy-4′-methylbenzophenone; 2,2′-dihydroxy-4-methoxybenzophenone; homomenthyl salicylate; 2-ethylhexyl 2-hydroxybenzoate; dimethicodiethylbenzalmalonate; 3-(4-(2,2-bis ethoxycarbonylvinyl)phenoxy)propenyl)methoxysiloxane/dimethylsiloxane copolymer; hexyl 2-(4′-diethylamino-2′-hydroxybenzoyl)benzoate; dioctylbutylamidotriazone (INCI: Diethylhexyl Butamidotriazone); 2,4-bis[5-1(dimethylpropyl)benzoxazol-2-yl-(4-phenyl)imino]-6-(2-ethylhexyl)imino-1,3,5-triazine with (CAS No. 288254-16-0); 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); tris(2-ethylhexyl) 4,4′,4″-(1,3,5-triazine-2,4,6-triyltriimino)trisbenzoate (also: 2,4,6-tris[anilino-(p-carbo-2′-ethyl-1′-hexyloxy)]-1,3,5-triazine (INCI: Ethylhexyl Triazone); 2,4,6-tribiphenyl-4-yl-1,3,5-triazine; merocyanine; titanium dioxide; zinc oxide.

The proportion of one or more UV filters is preferably selected in the range of more than 0.1% by weight up to 50% by weight, in particular in the range of 5 to 35% by weight, based on the total mass of the preparation.

The preparations according to the invention comprising one or more UV filters can be applied to wet or moist skin and thus achieve UV protection.

The use of the preparation comprising one or more UV filters is therefore suitable for application to wet skin, for drying the skin and at the same time protecting the skin from UV radiation.

Everything is done in one application step.

Even subsequent process steps, such as rinsing off and drying, do not result in loss of UV protection.

Surprisingly, when the preparation according to the invention is used to protect the skin from UV radiation, it is also possible to dispense with film formers.

### f) Stabilization, Viscosity Improvement

A disadvantage of various cosmetic or dermatological preparations is that they must have constant quality (visual, olfactory, microbial, etc.) over the storage and usage period. This also includes physical stability. The physical stability can often be achieved by increasing the viscosity and the associated reduced diffusion of the oil droplets. In the prior art, there are many examples of how, for example in an O/W emulsion, the viscosity of the outer phase is increased, the diffusion of the oil droplets is reduced and the long-term stability is thus increased. This is not possible in a water-free formulation. Furthermore, increasing the viscosity of lipophilic phases is more difficult since fewer mechanisms are available here. In general, polymeric oil thickeners or solids are then added. In accordance with the invention, the addition of wax components, in particular two different waxes, is preferably selected to improve the viscosity. Surprisingly, the combination of two waxes here leads to a significantly greater effect than the same concentration of the individual wax components.

In a further preferred embodiment, hydrogenated castor oil in combination with magnesium stearate are added to the preparations according to the invention.

The combination improves the stability of the preparation once again by a distinctly more efficient increase in viscosity. Whereas the respective individual components do not achieve this to the same extent.

In a comparative study, the addition of 3% by weight magnesium stearate does not cause any thickening effects or viscosity of the preparations according to the invention.

The addition of 3% by weight hydrogenated castor oil led to an increase in the viscosity of the preparation to 3400 mPas (25° C.).

The addition of 1.5% by weight magnesium stearate and 1.5% by weight hydrogenated castor oil, however, surprisingly resulted in a viscosity of the preparations according to the invention of up to 11,700 mPas (25° C.).

This means that the combination leads to a synergistic effect that increases the viscosity, and thus the stability, significantly more efficiently. This enables a much finer and broader spectrum of tailor-made sensory properties, since waxes generally have a strong influence on the sensory feel.

Therefore, preferably 0.2% by weight to 2% by weight hydrogenated castor oil and advantageously additionally 0.2 to 2% by weight magnesium stearate are added to the preparations according to the invention, based in each case on the total mass of the preparation.

In this case, the ratio by weight of the two substances, hydrogenated castor oil to magnesium stearate, is preferably selected between 0.75: 1.25 to 1.25: 0.75.

### f) Siloxane Elastomers

In a further preferred embodiment, the preparation according to the invention comprises one or more siloxane elastomers. One or more elastomers can preferably be selected in the proportion range from 0.1% by weight to 12% by weight, based on the total mass of the preparation.

Surprisingly, the drying behavior is further improved by adding one or more siloxane elastomers.

In a comparative study, it has been shown that adding siloxane elastomer and dimethicone to the preparations according to the invention improves the drying kinetics although there is no water absorption by the dimethicones.

Two preparations according to the invention, which differed only in that one preparation comprises a siloxane elastomer and the other preparation does not comprise an elastomer, were compared by a panel of ten experts under standardized conditions.

| M 685 | N 701 | INCI |
|---|---|---|
| 5.12 | 7.21 | Isoamyl Laurate |
| 5 | 5 | Carboxymethylcellulose |
| 3 | 3 | Hydrogenated Castor Oil |
| 15.74 | 22.25 | Isopropyl palmitate |
| 1.67 | 2.37 | Octyldodecanol |
| 1.67 | 2.37 | C12-15 Alkyl Benzoate |
| 20 | 0 | Dimethicone |
| 1 | 1 | Diisostearoyl Polyglyceryl-3 Dimer Dilinoleate |
| 1 | 1 | Polyglyceryl-4 |
| | | Diisostearate/Polyhydroxystearate/Sebacate |
| 5 | 5 | Calcium Starch Octenylsuccinate + |
| | | Aqua + Calcium Chloride |
| 0 | 10 | Dimethicone + Si-elastomer (Dimethicone |
| | | Crosspolymer) |
| 0.8 | 0.8 | Perfume |
| 6 | 6 | Glycerol |
| 4 | 4 | Magnesium Stearate |
| 30 | 30 | Alcohol Denat. + Aqua |

Result of the panel: The majority of people found N701 to be significantly drier than M685

### g) Skin Moisturizers

One or more skin moisturizers can be added to the preparations according to the invention. It has been found that the addition of skin moisturizers can further improve the sensory, haptic and, above all, the care properties of the preparations according to the invention.

Advantageously, 1% by weight up to 30% by weight, in particular 1% by weight to 20% by weight, based on the total mass of the preparation, of one or more skin moisturizers are added to the preparations. Glycerin is advantageously selected as the moisturizer.

Choosing a high proportion of glycerin in the range of up to 30% by weight has the advantage that high amounts of glycerin produce a warming effect that can be pleasant for the user.

### h) Other Substances Additionally Present

The cosmetic or dermatological preparations according to the invention may also contain cosmetic auxiliaries and other active ingredients as are customarily used in such preparations, e.g. substances to prevent foaming, dyes and color pigments, moisturizing and/or moisture-retaining substances, fats, oils, waxes or other common constituents of a cosmetic or dermatological formulation such as alcohols, polyols, polymers, foam stabilizers, electrolytes, organic solvents or silicone derivatives, self-tanners, buffers, pH regulators, herbal extracts, sebum-absorbing substances, UV filters, active ingredients such as anti-aging, anti-cellulite, anti-acne, anti-rosacea, anti-neurodermatitis, antioxidants, moisturizers, chelating agents, antiperspirants, bleaching and coloring agents, perfume, etc. provided that the additive does not hinder the properties required in terms of water absorption, stability and sensory properties or are excluded according to the invention.

The following can preferably be added to the preparations according to the invention as possible additional substances:
1. Volatile substances, such as in particular ethanol, cyclomethicones, short-chain dimethicones, short-chain paraffins, isodecyl neopentanoate, dibutyl adipate and other substances with a comparable volatility profile

Advantage: the additive results in a lighter skin feel, the possibility of controlling the sensory feel and a better perceived dryness of the consumer's skin
1. Sensory additives, in particular carbohydrates, for example starch or cellulose derivatives, silicone elastomers and powdery raw materials, fillers (polymeric and non-polymeric) which do not fall under the group of superabsorbents.

Advantages: the addition results in a more pleasant sensory feel and the possibility of controlling the sensory properties.
2. Oil thickeners

Advantages: the additive results in an increase in viscosity so that, for example, the preparation does not run through the user's fingers or has increased long-term stability.
3. Film formers

Advantages: the addition improves the water resistance. In the case of light protection preparations in particular, this is an essential property for the users so that they do not have to constantly re-apply cream after bathing. However, it is also possible in another embodiment in accordance with the invention to dispense entirely with film formers.

### Application Forms

As possible application forms, all common forms are initially suitable for the preparations according to the invention, as a result of which the preparations according to the invention offer a further formulation advantage in production.

Liquid, gel-like and/or semi-solid forms are preferred.

### Use

The invention comprises the use of a topically applicable preparation comprising
a) one or more lipids,
b) at least one W/O emulsifier and
c) at least 0.05% by weight of one or more superabsorbents, based on the total mass of the preparation,
   for drying and simultaneous care of the skin.

The preparation according to the invention can be applied to the still wet or damp skin, distributed and thus results in drying and simultaneous care of the skin. Drying is not to be understood as drying out the skin, but merely reducing the moisture or wetness that was previously present on the skin.

Further advantageous uses according to the invention of the preparations according to the invention result from the substances optionally added.

The following examples elucidate preparations according to the invention. Unless otherwise stated, the numbers are percentages by weight based on the total mass of the preparation.

### Example Formulations:

| INCI | 1 | 2 | 3 | 4 | 5 | 6 | 7 | 8 |
|---|---|---|---|---|---|---|---|---|
| Silica Silylate | 0 | 1 | 0 | 2 | 1.5 | 0 | 1.5 | 1.5 |
| Sodium starch glycolate | 5 | 7 | 5 | 3 | 7 | 5 | 7 | 7 |
| Isoamyl Laurate | 7 | 7 | | 10 | | 6 | 7 | 7 |
| Hydrogenated Castor Oil | 3 | 3 | 2 | 3 | 4 | 3 | 3 | 3 |
| Magnesium Stearate | 4 | 4 | 3 | 4 | 5 | 4 | 4 | 4 |
| Octyldodecanol | | 2 | 5 | 1 | 2.5 | | 2 | 2 |
| C12-15 Alkyl Benzoate | 2.5 | 2 | 2.5 | 2 | 3 | 2.5 | 4 | 4 |
| Isopropyl palmitate | 25 | | 20 | | 25 | 20 | 22 | |
| Cyclomethicone | 5 | | 10 | | | | | |
| Isodecyl Neopentanoate | | | | 20 | | | | |
| C13-16 Isoparaffin | | | | | 10 | | | |
| Simmondsia Chinensis Seed Oil | 10 | | | | | 10 | | |
| PPG-15 Stearyl Ether | | | | | | | 10 | |
| Butylene Glycol Dicaprylate/Dicaprate | | | | | | | | 22 |
| Paraffinum Liquidum | | 20 | | | | | | |
| Diisostearoyl Polyglyceryl-3 Dimer | 3 | 1 | 2 | 1 | 1 | 2 | 1 | 1 |
| Dilinoleate | | | | | | | | |
| Polyglyceryl-4 | 3 | 1 | 2 | 1 | 1 | 2 | 1 | 1 |
| Diisostearate/ | | | | | | | | |
| Polyhydroxystearate/Sebacate | | | | | | | | |
| Calcium Starch Octenylsuccinate + Aqua + | 6 | 2 | 5 | 4 | 5 | 7 | 2 | 2 |
| Calcium Chloride | | | | | | | | |
| Dimethicone + Dimethicone | | 10 | | | | 10 | 10 | 10 |
| Crosspolymer | | | | | | | | |
| Perfume | 0.8 | 0.25 | 0.7 | 0.3 | 0.8 | 0.5 | 1 | 1 |
| Glycerol | 5 | 5 | 0 | 10 | 8 | 6 | 3 | 3 |
| Alcohol Denat. + Aqua | to 100 | to 100 | to 100 | to 100 | to 100 | to 100 | to 100 | to 100 |

Example Formulations with UV Filters

| | 1 | 2 | 3 | 4 | 5 | 6 | 7 | 8 | 9 |
|---|---|---|---|---|---|---|---|---|---|
| Silica Silylate | 2 | 1 | 2 | 2 | 3 | 0 | 2 | 0.5 | 1 |
| Sodium starch glycolate | 5 | 4 | 0 | 4 | 8 | 6 | 2 | 5 | 3 |
| Isoamyl Laurate | 0 | 0 | 0 | 0 | 0 | 0 | 0 | 0 | 0 |
| Hydrogenated Castor Oil | 3 | 3 | 3 | 3 | 3 | 3 | 3 | 3 | 3 |
| Magnesium Stearate | 0 | 0 | 0 | 0 | 0 | 0 | 0 | 0 | 0 |
| Octyldodecanol | 1 | 0 | 2 | 0 | 0 | 0 | 4 | 0 | 0 |
| Isopropyl palmitate | 9.5 | 0 | 0 | 0 | 0 | 0 | 0 | 0 | 0 |
| Dibutyl Adipate | 3 | 3 | 3 | 3 | 3 | 3 | 10 | 3 | 3 |
| C12-15 Alkyl Benzoate | 0 | 9.5 | 3 | 0 | 0 | 0 | 0 | 0 | 0 |
| Cyclomethicone | 0 | 0 | 0 | 0 | 0 | 9.5 | 0 | 0 | 0 |
| Isodecyl Neopentanoate | 0 | 0 | 0 | 0 | 0 | 0 | 0 | 12 | 0 |
| C13-16 Isoparaffin | 0 | 0 | 0 | 0 | 0 | 0 | 0 | 0 | 10 |
| Simmondsia Chinensis | 0 | 0 | 0 | 0 | 7 | 0 | 0 | 0 | 0 |
| Seed Oil | | | | | | | | | |
| PPG-15 Stearyl Ether | 0 | 0 | 0 | 9 | 0 | 0 | 0 | 0 | 0 |
| Butylene Glycol | 3 | 3 | 10 | 3 | 3 | 3 | 3 | 3 | 3 |
| Dicaprylate/Dicaprate | | | | | | | | | |
| Cera Alba | 3 | 3 | 3 | 3 | 3 | 3 | 3 | 3 | 3 |
| Diisostearoyl | 0.5 | 0.5 | 0.5 | 0.5 | 0.5 | 0.5 | 0.5 | 0.5 | 0.5 |
| Polyglyceryl-3 Dimer | | | | | | | | | |
| Dilinoleate | | | | | | | | | |
| Polyglyceryl-4 | 0.5 | 0.5 | 0.5 | 0.5 | 0.5 | 0.5 | 0.5 | 0.5 | 0.5 |
| Diisostearate/ | | | | | | | | | |
| Polyhydroxystearate/ | | | | | | | | | |
| Sebacate | | | | | | | | | |
| Calcium Starch | 4 | 5 | 5 | 5 | 3 | 7 | 5 | 5 | 5 |
| Octenylsuccinate + Aqua + | | | | | | | | | |
| Calcium Chloride | | | | | | | | | |
| Dimethicone + | | 5 | | 5 | | 5 | 5 | | |
| Dimethicone | | | | | | | | | |
| Crosspolymer | | | | | | | | | |
| Perfume | 0.3 | 0.7 | 1 | 0.5 | 0.7 | 0.6 | 9 | 0.7 | 0.3 |
| Glycerol | 2 | 2.5 | 3 | 2.5 | 7 | 2.5 | 6 | 2.5 | 4 |
| Alcohol Denat. + Aqua | to 100 | to 100 | to 100 | to 100 | to 100 | to 100 | to 100 | to 100 | to 100 |
| Homosalate | 0 | 0 | 0 | 0 | 9 | 9 | 0 | 9 | 0 |
| Octocrylene | 0 | 0 | 8 | 0 | 9 | 9 | 0 | 9 | 0 |
| Ethylhexyl Salicylate | 2 | 4.5 | | 5 | 4.5 | 4.5 | 4.5 | 4.5 | 4.5 |
| Bis-Ethylhexyloxyphenol | 3.5 | 2 | 3.5 | 2.5 | 2 | 2 | 2 | 2 | 2 |
| Methoxyphenyl Triazine | | | | | | | | | |
| Butyl Methoxydibenzoylmethane | 4.8 | 4 | 4.5 | 3 | 4.5 | 4.5 | 4 | 4.5 | 4 |
| Polysilicone-15 | 0 | 0 | 0 | 0 | 1 | 0 | 0 | 1 | 0 |
| Diethylamino Hydroxybenzoyl | 1 | 0 | 0 | 0 | 0 | 0 | 0 | 0 | 0 |
| Hexyl Benzoate | | | | | | | | | |
| Ethylhexyl Triazone | 0.75 | 1.5 | 0 | 2 | 0 | 0 | 1.5 | 0 | 1.5 |
| Titanium Dioxide (nano) | 2 | 0.5 | 6 | 1 | 0 | 0 | 3 | 0 | 3 |
| Phenylbenzimidazole Sulfonic | 0 | 1 | 0 | 0 | 0 | 0 | 1 | 5 | 1 |
| Acid | | | | | | | | | |
| Zinc Oxide | 0 | 0 | 0 | 0 | 0 | 0 | 3 | 0 | 3 |

| INCI | 10 |
|---|---|
| Silica Silylate | 2 |
| Carboxymethyl starch (sodium starch glycolate) | 5 |
| Dibutyl Adipate | 3 |
| C12-15 Alkyl Benzoate | 9.5 |
| Hydrogenated Castor Oil | 3 |
| Butylene Glycol Dicaprylate/Dicaprate | 3 |
| Cera Alba | 3 |
| Diisostearoyl Polyglyceryl-3 Dimer Dilinoleate | 0.5 |
| Polyglyceryl-4 Diisostearate/Polyhydroxystearate/Sebacate | 0.5 |
| Calcium Starch Octenylsuccinate + Aqua + Calcium Chloride | 5 |
| Dimethicone + Dimethicone Crosspolymer | 5 |
| Perfume | 0.7 |
| Glycerol + Aqua | 2.5 |
| Alcohol Denat. + Aqua | 27.3 |
| Homosalate | 9 |
| Octocrylene | 9 |
| Ethylhexyl Salicylate | 4.5 |
| Bis-Ethylhexyloxyphenol Methoxyphenyl Triazine | 2 |
| Butyl Methoxydibenzoylmethane | 4.5 |
| Polysilicone-15 | 1 |

1.-18. (canceled)

19. A cosmetic or dermatological preparation, wherein the preparation comprises
(a) one or more lipids,
(b) one or more W/O emulsifiers,
(c) at least 0.05% by weight of one or more superabsorbents, based on the total mass of the preparation,
and wherein the preparation comprises a maximum of 1% by weight of water, based on a total mass of the preparation.

20. The preparation of claim 19, wherein the preparation consists of components (a) to (c) and a maximum of 1% by weight of water.

21. The preparation of claim 19, wherein the one or more lipids have a spreading value above 400.

22. The preparation of claim 19, wherein the one or more lipids are selected from dibutyl adipate, isopropyl myristate, isodecyl neopentanoate, octyldodecanol, caprylic/capric triglycerides, propylene glycol dicaprylate/dicaprate, dicaprylyl ether, paraffinum liquidum, isopropyl palmitate, C13-16 isoparaffin, isohexadecane, cyclomethicone, hydrogenated polydecene, octyldodecyl myristate, decyl oleate, butylene glycol dicaprylate/dicaprate, dicaprylyl carbonate, hydrogenated polyisobutene, isopropyl isostearate, cetearyl isononanate, isopropyl stearate, phenyl trimethicone, C12-15 alkyl benzoate, ethylhexyl cocoate, squalane, ethylhexyl stearate, cetearyl ethylhexanoate+isopropyl myristate, C12-13 alkyl lactate, coco-caprylate/caprate, Persea gratissima oil, Simmondsia chinensis seed oil.

23. The preparation of claim 19, wherein the one or more lipids comprise one or more waxes having a melting point in a range of from 30° C. to 120° C.

24. The preparation of claim 23, wherein a proportion of the one or more waxes is from 1% to 14% by weight, based on the total mass of the preparation.

25. The preparation of claim 19, wherein the preparation comprises from 20% to 75% by weight of (a).

26. The preparation of claim 19, wherein the preparation comprises from 40% to 60% by weight of (a).

27. The preparation of claim 19, wherein the one or more W/O emulsifiers are selected from polyglyceryl-n diisostearates and polyglyceryl-n dipolyhydroxystearates.

28. The preparation of claim 19, wherein the one or more W/O emulsifiers are selected from polyglyceryl-n dipolyhydroxystearates, diisostearoyl polyglyceryl-n dimer dilinoleates, and polyglyceryl-n diisostearates/polyhydroxystearates/sebacates.

29. The preparation of claim 19, wherein the preparation comprises from 0.25% to 10% by weight of (b).

30. The preparation of claim 19, wherein the one or more superabsorbents are selected from carboxymethyl celluloses and carboxymethyl starches.

31. The preparation of claim 19, wherein the one or more superabsorbents comprise sodium starch glycolate and/or sodium cellulose glycolate.

32. The preparation of claim 19, wherein the preparation comprises from 0.05% to 20% by weight of (c).

33. The preparation of claim 19, wherein the preparation comprises from 0.1% to 10% by weight of (c).

34. The preparation of claim 19, wherein the preparation further comprises one or more UV filters.

35. The preparation of claim 19, wherein the preparation further comprises hydrogenated castor oil and magnesium stearate.

36. The preparation of claim 19, wherein the preparation further comprises one or more siloxane elastomers.

37. The preparation of claim 19, wherein the preparation further comprises one or more substances selected from ethanol, cyclomethicones, short-chain dimethicones, short-chain paraffins, isodecyl neopentanoate, dibutyl adipate.

38. A method of drying and simultaneously caring for skin, wherein the method comprises applying to wet skin the preparation of claim 19.
